(19) United States
(12) Plant Patent Application Publication
(10) Pub. No.: US 2004/0139510 P1
(43) Pub. Date: Jul. 15, 2004

(54) BIDENS FERULIFOLIA PLANT NAMED 'BIDANTIS'

(76) Inventor: Anna M.W.P. Houbraken, Enkhuizen (NL)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/339,804

(22) Filed: Jan. 10, 2003

(51) Int. Cl. A01H 5/00

(52) U.S. Cl. PLT/263

(57) ABSTRACT

A distinct cultivar of Bidens plant named ‘Bidantis’ which is particularly distinguished by its vigorous character, upright (bit trailing) plant habit, dark green foliage and golden yellow flowers. The new cultivar can perfectly be used in mixed hanging baskets.

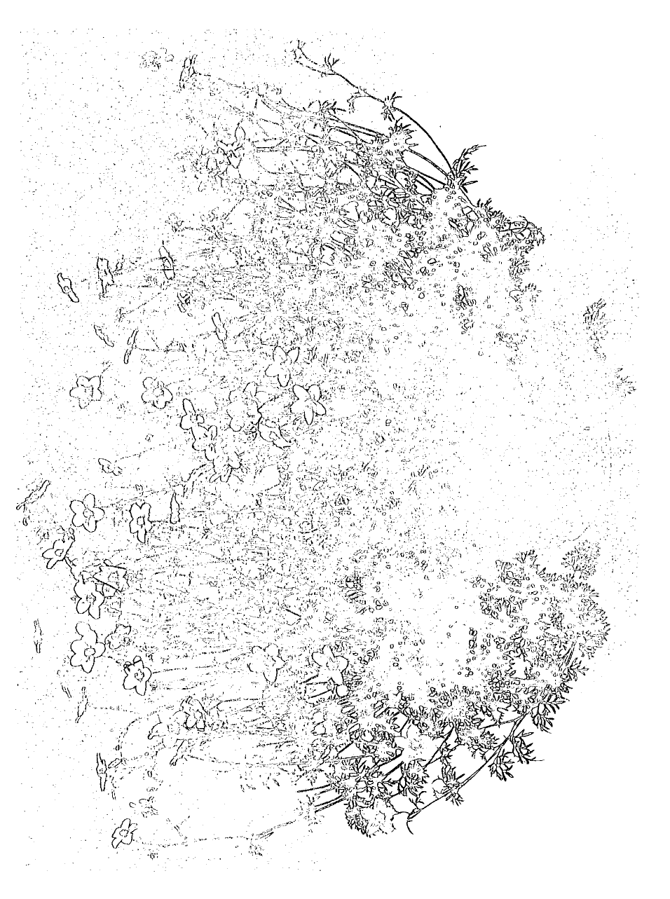

### VARIETAL DENOMINATION

‘Bidantis’

### BACKGROUND OF THE NEW PLANT

The present invention relates to a new distinct cultivar of Bidens, botanically known as Bidens ferulifolia, native to South USA and Mexico. The new culivar is propagated by cuttings resulting from a seedling from Bidens ferulifolia cv. ‘BQ001’ (not patented). This new plant is an annual. The new cultivar is a product of a planned breeding program conducted by the inventor in Enkhuizen, The Netherlands. The objective of the breeding program was to develop an intermediate between the compact Bidens varieties and the very vigorous trailing Bidens varieties. As a result of an open pollination of Bidens ferulifolium BQ001 (A 312-2) (unpatented and commercialised by Syngenta Seeds BV in Europe) the present cultivar was created in 1999 in Enkhuizen. ‘Bidantis’ was asexually reproduced by cuttings for 3 years now and trialled under different conditions in Europe on Syngenta trial ground properties. It has been found to retain its distinctiveness characteristics through successive propagation, and its novelty appears to be firmly fixed.

### DESCRIPTION OF THE DRAWING

This new Bidens plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Bidens plant. The data which defines these characteristics were collected from asexual reproductions carried out in Enkhuizen, Netherlands. The plant history was taken on 13 weeks old plants, grown in hanging baskets in Enkhuizen, The Netherlands with day temperatures ranging from 16 and 20 degrees C., and night temperatures ranging from 12 to 14 degrees C. Color references are primarily to the RHS Color Chart of The Royal Horticultural Society of London.

Differences between the new cultivar
‘Bidantis’ and two similar cultivars

| | | | ‘Solaire | | | |
|---|---|---|---|---|---|---|
| | ‘Bidantis’ | | Compact’ | | ‘Peter's Goldteppich’ | |
| Plant Height | 30-35 | cm | 20 | cm | 12 | cm |
| Plant Width | 70-80 | cm | 90 | cm | 100 | cm |
| Flower Color | More Golden | | 12A | | 12A | |
| | than 9A | | | | | |
| Flower Size | 3-3.5 | cm | 3.4 | cm | 4-5 | cm |

The plant:

Classification.—Botanical: Bidens ferulifolia cv. ‘Bidantis’.

Parentage:

Female parent.—Seedling selection of Bidens ferulifolia cv. ‘BQ001’, which is not patented or commercialized by Syngenta Seeds; Breeders code of the new plant is B 74-3.

Male parent.—Unidentified selection of Bidens ferulifolia.

Propagation:

Type cuttings.—Terminal cuttings.

Time to intiated roots.—10-12 days at air temperature of 21 degrees C.

Time to develop roots.—14-18 days at air temperature of 17 degrees C.

Plant description:

Growth habit.—Upright and little trailing.

Plant height.—30-35 cm.

Vigor.—Much more vigorous than ‘Bidtis 1’ and ‘Peter's Goldteppich’.

Spreading area of plant.—70-80 cm.

Strength.—Resistant to hot and cold weather.

Branching character.—Freely branching and lateral branching at every node.

Number of branches per plant.—7 branches/plant.

Crop time.—12 weeks.

The stem:

Diameter.—1-2 mm.

Shape.—Cylindrical.

Anthocyan pigmentation.—Present in older stems.

Length of internode.—4-7 cm depending on light conditions.

Pubescence.—Absent.

Color.—Young stem: 146A — Older stem: 152A with some anthocyan pigmentation.

The foliage:

Arrangement.—Opposite, but alternate on flower stems.

Shape of leaf.—2-pinnate fern-like leaves.

Leaf apex.—Apiculate.

Leaf base.—Acute.

Texture.—Smooth young leaves.

Leaf length.—2.5-3.5 cm.

Leaf width.—1.5-2.5 cm.

Depth of incision.—Fully incised.

Color.—Upper side: Young leaf 146B Old leaf 137A — Lower side: Young leaf 147B Old leaf 147B.

Pubescence.—Little hairy.

Petiole.—Length: 0.5-1.0 cm — Color: 146A.

Venation.—Shape: Pinnate — Color: 146B.

The bud:

Peduncle length.—6-10 cm.

Size bud.—Diameter: 0.3-0.5 cm — Length: 0.5-1.0 cm.

Shape bud.—Oblong.

Color bud.—14A.

Inflorescence:

Diameter.—3-3.5 cm.

Form.—Capitulum with 1 series of ray florets and more series of disc florets.

Overlapping of ray florets.—Separate ray florets, but little overlap in flowerhead

Receptacle.—Shape: Brush-shape — Height 4 mm — Color: 147B.

Involucre.—8 bracts in 1 series — Color: 144A.

Fragrance.—Weak.

Number of flowers per plant.—±70 flowers per plant depending on growth conditions and age of plant.

Lastingness of flowers.—7 days.

Ray florets:

No. of ray florets per inflor.—5.

Size.—Length: 17-20 mm — Width: 11-13 mm.

Shape of florets.—Ovate with 2 light furrows.

Color.—More bright and intense golden yellow than 9A.

Disc florets:

No. of disc florets per inflor.—±70 disc florets/inflorescence.

Size.—Length: 7 mm — Width: 1 mm.

Shape of florets.—Tubular.

Color.—13A.

Androecium:

Anther color.—200A.

Pollen color.—17A.

Amount of pollen.—Few.

Gynoecium:

Pistil number.—±70 pistils/inflorescence.

Syle color.—17B.

Stigma color.—17B.

Seed:

Production.—Yes.

Size.—0.5-0.7 cm.

Color.—202A.

Shape.—2 rigid awns with minute reflexed points on the achene.

Disease resistance and tolerance for weather conditions: Good tolerance to heat. Strong resistance to pests and diseases.

What is claimed is:

1. A new and distinct variety of Bidens plant, substantially as herein illustrated and described, characterized particularly as to novelty by its upright vigorous plant habit with bright golden yellow flowers.
